SEQUENCE LISTING (55 sequences)

SEQ ID NO: 1 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 2 - protein, 608 aa, Homo sapiens
MDAMKRGLCCVLLLCGAVFVSPGADPVKPSRGPLVTCTCESPHCKGPTCRGAWCTVVLVREEGRHPQEHRGCGNLHRALCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGKPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQEEMTKNQVSLTCLVKGFYPSDIAVEWESNGQPENNYKTTPPVLDSDGPFFLYSKLTVDKSRWQQ

SEQ ID NO: 3 - protein, 260 aa, Homo sapiens
MDAMKRGLCCVLLLCGAVFVSPGADPVKPSRGPLVTCTCESPHCKGPTCRGAWCTVVLVREEGRHPQEHRGCGNLHRALCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSR

SEQ ID NO: 4 - protein, 9 aa, Homo sapiens
QDDCTGDED

SEQ ID NO: 5 - protein, 9 aa, Homo sapiens
RKRGLKLAT

SEQ ID NO: 6 - protein, 11 aa, Homo sapiens
HRDFSAEYEED

SEQ ID NO: 7 - protein, 15 aa, Homo sapiens
EQIQDDCTGDEDYTY

SEQ ID NO: 8 - protein, 16 aa, Homo sapiens
GQDDCTGDEDYTYLIL

SEQ ID NO: 9 - protein, 9 aa, Homo sapiens
QDDCTGDED

SEQ ID NO: 10 - protein, 8 aa, Homo sapiens
KFIIPSPK

SEQ ID NO: 11 - protein, 9 aa, Homo sapiens
VKAQYKKNK

SEQ ID NO: 12 - protein, 9 aa, Homo sapiens
RKRGLKLAT

SEQ ID NO: 13 - protein, 16 aa, Homo sapiens
LSVKAQYKKNKHRHSI

SEQ ID NO: 14 - protein, 15 aa, Homo sapiens
FILKTSQCTLKEVYG

SEQ ID NO: 15 - protein, 15 aa, Homo sapiens
GNVATEISTERDLGQ

SEQ ID NO: 16 - protein, 15 aa, Homo sapiens
KKMGLAFESTKSTSP

SEQ ID NO: 17 - protein, 15 aa, Homo sapiens
VTYLVALIPEPSAQQ

SEQ ID NO: 18 - protein, 15 aa, Homo sapiens
KPSLMIQKAAIQALR

SEQ ID NO: 19 - protein, 15 aa, Homo sapiens
PTVMDFRKFSRNYQL

SEQ ID NO: 20 - protein, 15 aa, Homo sapiens
KVLVDHFGYTKDDKH

SEQ ID NO: 21 - protein, 15 aa, Homo sapiens
PTGAGLQLQISSSGV

SEQ ID NO: 22 - protein, 15 aa, Homo sapiens
STTKTEVIPPLIENR

SEQ ID NO: 23 - protein, 15 aa, Homo sapiens
YNRQSMTLSSEVQIP

SEQ ID NO: 24 - protein, 15 aa, Homo sapiens
TVSKRVAWHYDEEKI

SEQ ID NO: 25 - protein, 15 aa, Homo sapiens
GLPDFHIPENLFLKS

SEQ ID NO: 26 - protein, 15 aa, Homo sapiens
SGGNTSTDHFSLRAR

SEQ ID NO: 27 - protein, 15 aa, Homo sapiens
VKEVKIDGQFRVSSF

SEQ ID NO: 28 - protein, 15 aa, Homo sapiens
DMTFSKQNALLRSEY

SEQ ID NO: 29 - protein, 15 aa, Homo sapiens
AKFSLDGKAALTELS

SEQ ID NO: 30 - protein, 14 aa, Homo sapiens
KYNALDLTNNGKRL

SEQ ID NO: 31 - protein, 15 aa, Homo sapiens
AHTNGNGKLALWGEH

SEQ ID NO: 32 - protein, 15 aa, Homo sapiens
DLTLLDSPIKVPLLL

SEQ ID NO: 33 - protein, 15 aa, Homo sapiens
NDYLNSFNWERQVSH

SEQ ID NO: 34 - protein, 15 aa, Homo sapiens
HLFIENIDFNKSGSS

SEQ ID NO: 35 - protein, 15 aa, Homo sapiens
FRAKVHELIERYEVD

SEQ ID NO: 36 - protein, 15 aa, Homo sapiens
REVTQRLNGEIQALE

SEQ ID NO: 37 - protein, 15 aa, Homo sapiens
SDWWTLAAKNLTDFA

SEQ ID NO: 38 - protein, 15 aa, Homo sapiens
IPSFTIDFVEMKVKI

SEQ ID NO: 39 - protein, 15 aa, Homo sapiens
QSPLFTLDANADIGN

SEQ ID NO: 40 - protein, 15 aa, Homo sapiens
GVIVKINNQLTLDSN

SEQ ID NO: 41 - protein, 15 aa, Homo sapiens
GSLNFSKLEIQSQVD

SEQ ID NO: 42 - protein, 15 aa, Homo sapiens
SWQVSARFNQYKYNQ

SEQ ID NO: 43 - protein, 15 aa, Homo sapiens
LCEFISQSIKSFDRH

SEQ ID NO: 44 - protein, 15 aa, Homo sapiens
PSLELPVLHVPRNLK

SEQ ID NO: 45 - protein, 15 aa, Homo sapiens
EGSHNSTVSLTTKNM

SEQ ID NO: 46 - protein, 15 aa, Homo sapiens
ASEANTYLNSKSTRS

SEQ ID NO: 47 - protein, 15 aa, Homo sapiens
ALNANTKNQKIRWKN

SEQ ID NO: 48 - protein, 15 aa, Homo sapiens
DKFIIPGLKLNDLNS

SEQ ID NO: 49 - protein, 15 aa, Homo sapiens
IAALDLNAVANKIAD

SEQ ID NO: 50 - protein, 15 aa, Homo sapiens
ASKTKGTFAHRDFSA

SEQ ID NO: 51 - protein, 15 aa, Homo sapiens
DEETQIKVNWEEEAA

SEQ ID NO: 52 - protein, 15 aa, Homo sapiens
LTQEGQASFQGLKDN

SEQ ID NO: 53 - protein, 15 aa, Homo sapiens
ADVISMYRELLKDLS

SEQ ID NO: 54 - protein, 15 aa, Homo sapiens
ASQELQQIHQYIMAL

SEQ ID NO: 55 - protein, 15 aa, Homo sapiens
IKSQAIATKKIISDY

(19) United States
(12) Patent Application Publication
(10) Pub. No.: US 2021/0403549 A1
(43) Pub. Date: Dec. 30, 2021

(54) COMPOUNDS AND COMPOSITIONS THAT INHIBIT OR PREVENT LIPOPROTEIN ENTRY INTO THE ENDOTHELIUM

(71) Applicant: YALE UNIVERSITY, New Haven, CT (US)

(72) Inventors: William C. SESSA, Madison, CT (US); Jan KRAEHLING, Düsseldorf (DE)

(21) Appl. No.: 16/614,225

(22) PCT Filed: May 17, 2018
(86) PCT No.: PCT/US18/33142

§ 371 Date: Nov. 15, 2019

Related U.S. Application Data
(60) Provisional application No. 62/507,452, filed on May 17, 2017.

(51) Int. Cl. C07K 16/24; A61K 45/06; C07K 14/705

(52) U.S. Cl. CPC C07K 16/247; A61K 45/06; C07K 2319/30; C07K 2317/32; C07K 2317/76; C07K 14/705

(57) ABSTRACT

In one aspect, the invention comprises a fusion construct comprising ALKI extracellular domain and a stability enhancing domain. In another aspect, the invention comprises an antibody that interferes with or blocks the ALKI extracellular domain binding to LDL, but does not interfere with or block the ALKI extracellular domain binding to BMP9/10. In yet another aspect, the invention comprises methods of reducing rates of LDL uptake in the endothelial cells of a patient in need thereof by administering the fusion construct or monoclonal antibodies described herein to the patient.

### CROSS REFERENCE TO RELATED APPLICATIONS

The present application claims priority under 35 U.S.C. §119(e) to U.S. Provisional Patent Application No. 62/507,452 filed May 17, 2017, which is incorporated herein by reference in its entirety.

### STATEMENT REGARDING FEDERALLY SPONSORED RESEARCH OR DEVELOPMENT

This invention was made with government support under HL061371 awarded by National Institutes of Health. The government has certain rights in the invention.

### BACKGROUND OF THE INVENTION

Atherosclerotic cardiovascular disease or atherosclerosis is the leading cause of death worldwide. Atherosclerosis is initiated by subendothelial retention of lipoproteins. In humans, low density lipoprotein (LDL) is the main carrier of cholesterol to peripheral organs, and is considered to be the major lipoprotein responsible for atherogenesis. LDL from the blood stream must cross the endothelium to reach the subendothelial area, where it accumulates and oxidizes over time. As atherosclerosis correlates tightly with plasma LDL concentrations, plaque formation will occur in patients with elevated LDL. Eventually the established plaques can undergo sudden ruptures, triggering clots within the artery opening and causing cardiovascular disease, stroke, and other vascular disease complications.

There is thus a need in the art for novel compositions and methods that can be used to prevent, minimize or revert LDL uptake through the endothelium. The present disclosure addresses this need.

### SUMMARY OF THE INVENTION

In one aspect, the invention provides a fusion construct comprising a polypeptide with greater than 70% sequence identity to the ALK1 extracellular domain (SEQ ID NO:1), which is fused to a stability enhancing domain, wherein the fusion construct does not bind BMP9 and/or BMP10.

In various embodiments, the polypeptide has greater than 90% sequence identity to the ALK1 extracellular domain.

In various embodiments, wherein the ALK1 extracellular domain comprises a point mutation in E75 and/or H73.

In various embodiments, the ALK1 extracellular domain comprises at least one point mutation selected from the group consisting of E75A, E75F and E75V.

In various embodiments, the ALK1 extracellular domain comprises at least one point mutation selected from the group consisting of H73G, H73A and H73D.

In various embodiments, the stability enhancing domain comprises albumin, thioredoxin or a Fc region of an antibody.

In various embodiments, the antibody is IgG1.

In various embodiments, the invention provides a pharmaceutical composition comprising the fusion construct described herein and at least one pharmaceutically acceptable carrier.

In various embodiments, the invention provides a method of reducing, preventing or reversing LDL uptake into endothelial cells of a subject, the method comprising administering to the subject a fusion construct or a pharmaceutical composition as described herein.

In various embodiments, the subject is further administered an additional treatment for cardiovascular disease.

In various embodiments, the additional treatment for cardiovascular disease comprises administration of at least one HMG-CoA inhibitor.

In various embodiments, the invention provides a method of treating a LDL-related disease or disorder in a subject, the method comprising administering to the subject a fusion construct or a pharmaceutical composition as described herein.

In various embodiments, the subject is further administered an additional treatment for cardiovascular disease.

In various embodiments, the additional treatment for cardiovascular disease comprises administration of at least one HMG-CoA inhibitor.

In various embodiments, the disease or disorder includes at least one selected from the group consisting of familial hypercholesterolemia (FH) and atherosclerosis.

In various embodiments, the subject is a mammal.

In various embodiments, the mammal is human.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIG. 1A is a schematic depicting LDL accumulation and cholesterol homeostasis. At physiological levels, LDL is internalized largely by the LDLR (left panel) whereas at elevated levels, LDL accumulates by an unknown pathway (right panel). FIG. 1B is a representation of the results from the RNAi screen. Results from genome-wide RNAi screen and follow-up screens are shaded differently.

FIG. 2A depicts a comparison of 3 lipoproteins after activin receptor-like kinase 1 (ALK1) knockdown.

FIG. 2B depicts surface plasmon resonance (SPR) experiments with immobilized LDL and control, LDLR and ALK1.

FIG. 2C depicts LDLR binding to LDL presaturated with ALK1.

FIG. 2D depicts ALK1 binding to LDL presaturated with LDLR.

FIG. 2E depicts ALK1 or bone morphogenetic protein (BMP) 9/ALK1 binding to immobilized LDL.

FIG. 3 is a graph illustrating uptake of LDL in endothelial cells (EC) in the presence of increasing concentrations of ALK1-fragment crystallizable region (Fc). 1 nM ALK1-Fc is equimolar to 500 ng/ml of LDL. Data represent the mean±SEM, n=3 experiments.

FIG. 4A depicts a Coomassie gel showing purity of ALK1^{ecto}-Fc obtained commercially compared to preparations of the invention (WT and E75A-mutant). FIG. 4B depicts bio-layer interferometry data showing binding of the E75A-mutant to LDL. FIG. 4C is a graph indicating that HeLa cells expressing wildtype ALK1 or the E75A-mutant take up significantly more fluorescently labeled LDL (DiI-LDL) than cells expressing GFP. FIG. 4D depicts gel data showing that the single point mutation E75A ablates the ability of the ALK1^{ecto}-Fc to inhibit BMP9-induced ALK1 signaling through phosphor-SMAD1/5.

FIG. 5 depicts ELISA data for 12 antibodies (Fab fragments) that have been selected against the BMP9/ALK1 complex.

FIG. 6 depicts mapping of ALK1 binding to ApoB100- In order to identify binding sites that mediate the interaction of ALK1 with the core LDL protein, Apo B100, peptide mapping experiments were performed. 4,563 different peptides (15 AA in length overlapped by 14AA) derived from the human ApoB100 sequence were arrayed and incubated with a control-Fc, and ALK1-Fc and binding events measurement by fluorescence. As seen in FIG. 6, ALK Fc binding was selectively found in 3 domains (Site 1, 4 and 5) whereas the control protein interacted with Sites 2 and 3 but not 1,4 and 5. The AA sequence of binding for site 1 is SEQ ID NO: 4 QDDCTGDED, site 4 is SEQ ID NO: 5 RKRGLKLAT and site 5 is SEQ ID NO: 6 HRDFSAEYEED.

FIG. 7 depicts structure function analysis of Site 1. Site 1 was interrogated as a major N-terminal binding site for ALK1 on ApoB 100. To do this, each of the natural amino acids were substituted at each position of the 15mer peptide SEQ ID NO: 7 EQIQDDCTGDEDYTY and a consensus binding motif of SEQ ID NO: 4 5DDCTGDED12 on ApoB100 was elucidated as seen in FIG. 7. Interestingly, charge inversion of D5, D6, D10, Ell and D12 blocked ALK1 binding, whereas charged residues at positions T8, G9, Y13, T14 and Y15 increased ALK1 binding.

FIG. 8 depicts development of a fluorescence polarization (Fp) assay of ALK1 binding to ApoB100 peptide Site 1. In order to measure binding affinities of ApoB100 to ALK1 and to facilitate screening molecules that block this interaction, a Fp assay was developed using recombinant ALK1-Fc and an HPLC purified fluorescein labelled peptide, -SEQ ID NO: 8 GQDDCTGDEDYTYLIL with G being an N-terminal linker to fluorescein and the C-terminal L amidated. As seen in FIG. 8, ALK-1 dose-dependently binds to the peptide with an estimated K_{d} of 2.45 micromolar but Fc alone did not bind.

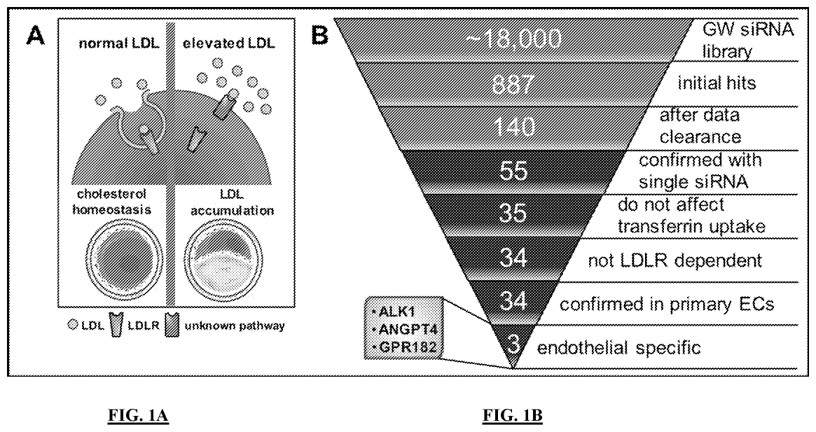

### DETAILED DESCRIPTION OF THE INVENTION

The present invention provides compounds that inhibit, minimize or reverse LDL endothelial uptake mediated by activin like kinase 1 (ALK1), which is a type I serine/threonine receptor kinase that belongs to the transforming growth factor (TGF)-β superfamily. Thus, the compounds of the invention can be used to treat diseases such as, but not limited to, familial hypercholesterolemia (FH) and/or atherosclerosis. In certain embodiments, the compound of the invention is an antibody that recognizes and specifically binds to the LDL binding site on ALK1. In other embodiments, the compound of the invention comprises an ALK1 extracellular domain polypeptide fused with a stability enhancing domain, such as but not limited to, IgG1-Fc.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

Generally, the nomenclature used herein and the laboratory procedures in biochemistry, immunology, cell culture, molecular genetics, pharmacology, and organic chemistry are those well-known and commonly employed in the art.

Standard techniques are used for biochemical and/or biological manipulations. The techniques and procedures are generally performed according to conventional methods in the art and various general references (e.g., Sambrook & Russell, 2012, Molecular Cloning, A Laboratory Approach, Cold Spring Harbor Press, Cold Spring Harbor, N.Y., and Ausubel, et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein the terms “alteration,” “defect,” “variation,” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

The term “antibody” as used herein refers to an immunoglobulin molecule that specifically binds with an antigen. Antibodies may be intact immunoglobulins derived from natural sources or from recombinant sources and may be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized, or can be derived from a biological sample. Such a biological sample can include, but is not limited to a tissue sample, a tumor sample, a cell or a biological fluid.

As used herein, “ALK1” or “ACVRL1” are used interchangeably and refer to the member of the human transforming growth factor superfamily having the polypeptide sequence of SEQ ID NO:1. As used herein, “extracellular domain” of ALK1 refers to amino acids 22-118 of ALK1.

| | |
|---|---|
| 1 | mtlgsprkgl lmllmalvtq gdpvkpsrgp lvtctcesph ckgptcrgaw ctvvlvreeg |
| 61 | rhpqehrgcg nlhrelcrgr ptefvnhycc dshlcnhnvs lvleatqpps eqpgtdgqla |
| 121 | lilgpvlall alvalgvlgl whvrrrqekq rglhselges slilkaseqg dsmlgdllds |
| 181 | dcttgsgsgl pflvqrtvar qvalvecvgk grygevwrgl whgesvavki fssrdeqswf |
| 241 | reteiyntvl lrhdnilgfi asdmtsrnss tqlwlithyh ehgslydflq rqtlephlal |
| 301 | rlavsaacgl ahlhveifgt qgkpaiahrd fksrnvlvks nlqcciadlg lavmhsqgsd |
| 361 | yldignnprv gtkrymapev ldeqirtdcf esykwtdiwa fglvlweiar rtivngived |
| 421 | yrppfydvvp ndpsfedmkk vvcvdqqtpt ipnrlaadpv lsglaqmmre cwypnpsarl |
| 481 | talrikktlq kisnspekpk viq |

As used herein, “ALK1-E75A-hsIgG-Fc” refers to a fusion construct comprising the extracellular domain of ALK1 with an E75A point mutation fused to a IgG fragment and having the polypeptide sequence of SEQ ID NO: 2.

| | |
|---|---|
| MDAMKRGLCC VLLLCGAVFV SPGADPVKPS RGPLVTCTCE SPHCKGPTCR GAWCTVVLVR | 60 |
| EEGRHPQEHR GCGNLHRALC RGRPTEFVNH YCCDSHLCNH NVSLVLEATQ PPSEQPGTDG | 120 |
| QLATGGGTHT CPPCPAPEAL GAPSVFLFPP KPKDTLMISR TPEVTCVVVD VSHEDPEVKF | 180 |
| NWYVDGVEVH NAKTKPREEQ YNSTYRVVSV LTVLHQDWLN GKEYKCKVSN KALPVPIEKT | 240 |
| ISKAKGQPRE PQVYTLPPSR EEMTKNQVSL TCLVKGFYPS DIAVEWESNG QPENNYKTTP | 300 |
| PVLDSDGPFF LYSKLTVDKS RWQQGNVFSC SVMHEALHNH YTQKSLSLSP GKPCPAPEAL | 360 |
| GAPSVFLFPP KPKDTLMISR TPEVTCVVVD VSHEDPEVKF NWYVDGVEVH NAKTKPREEQ | 420 |
| YNSTYRVVSV LTVLHQDWLN GKEYKCKVSN KALPVPIEKT ISKAKGQPRE PQVYTLPPSR | 480 |
| EEMTKNQVSL TCLVKGFYPS DIAVEWESNG QPENNYKTTP PVLDSDGPFF LYSKLTVDKS | 540 |
| RWQQEEMTKN QVSLTCLVKG FYPSDIAVEW ESNGQPENNY KTTPPVLDSD GPFFLYSKLT | 600 |
| VDKSRWQQ | 608 |

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

By the term “applicator,” as the term is used herein, is meant any device including, but not limited to, a hypodermic syringe, a pipette, and the like, for administering the compounds and compositions of the invention.

As used herein, the term “binding” refers to the adherence of molecules to one another, such as, but not limited to, enzymes or any other proteins to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound which is sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation.

The term “epitope” as used herein is defined as a small chemical molecule on an antigen that may elicit an immune response, inducing B and/or T cell responses. An antigen may have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

As used herein, “IgG 1-Fc” means immunoglobulin gamma-1 heavy chain constant region having the polypeptide sequence of SEQ ID NO:3

| | |
|---|---|
| MDAMKRGLCC VLLLCGAVFV SPGADPVKPS RGPLVTCTCE SPHCKGPTCR GAWCTVVLVR | 60 |
| EEGRHPQEHR GCGNLHRALC RGRPTEFVNH YCCDSHLCNH NVSLVLEATQ PPSEQPGTDG | 120 |
| QLATGGGTHT CPPCPAPEAL GAPSVFLFPP KPKDTLMISR TPEVTCVVVD VSHEDPEVKF | 180 |
| NWYVDGVEVH NAKTKPREEQ YNSTYRVVSV LTVLHQDWLN GKEYKCKVSN KALPVPIEKT | 240 |
| ISKAKGQPRE PQVYTLPPSR | 260 |

As used herein, the term “immunoglobulin” or “Ig” is defined as a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitor-urinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Parenteral” administration of a composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

By the term “specifically binds,” as used herein, is meant a molecule, such as an antibody, which recognizes and binds to another molecule or feature, but does not substantially recognize or bind other molecules or features in a sample.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.3, and 6. This applies regardless of the breadth of the range.

### Disclosure

The present invention provides compounds that inhibit, minimize or reverse LDL endothelial uptake mediated by ALK1. Thus, the compounds of the invention can be used to treat diseases such as, but not limited to, familial hypercholesterolemia (FH) and/or atherosclerosis. In certain embodiments, the compound of the invention is an antibody that recognizes and specifically binds to the LDL binding site on ALK1. In other embodiments, the compound of the invention comprises an ALK1 extracellular domain polypeptide fused with a stability enhancing domain, such as but not limited to, IgG1-Fc.

Endothelial cells have a LDL receptor (LDLR)-independent mechanism of LDL uptake. The pathways that define LDL entry into and across endothelium are not known at this time. Efforts were made to identify genes required for LDL entry into endothelial cells using a non-biased, genomic approach, whereby a high content imaging method was used to measure LDL entry into endothelial cells and a genome wide siRNA screen was used to identify gene candidates. By using this approach, ALK1 was identified as a novel LDL binding protein and shown to bind LDL directly.

As demonstrated herein, ALK1 mediates LDL uptake (endocytosis) and transcytosis independently of the LDLR by direct binding to LDL in a non-degradative pathway. The endogenous ALK1 ligands GDF2 (also known as BMP9) and BMP10 bind to a distinct domain on ALK1 than LDL, as demonstrated by SPR data. It is thus possible to inhibit or minimize LDL endothelial uptake by using a synthetic antibody that binds to the ALK1 LDL binding site, but yet does not bind to the BMP9/BMP10 binding site. It is further possible to inhibit or minimize LDL endothelial uptake by using a soluble decoy construct comprising the extracellular domain of ALK1 (amino acids 22-118) fused with a stabilizing group, such as but not limited to IgG1-Fc, which in certain embodiments extends the construct half-life in vivo. In certain embodiments, the soluble decoy construct has a single point mutation in the ALK1 extracellular domain (E75, such as but not limited to E75A, E75F or E75V; and/or H73, such as but not limited to H73G, H73A, or H73D), which ablates its ability to bind BMP9/BMP10 thus preventing unwanted effects on angiogenesis.

### Fusion Constructs

In one aspect, the invention comprises a soluble fusion construct comprising the ALK1 extracellular domain and a stability enhancing domain. In certain embodiments, the fusion construct acts as a decoy receptor that is useful as a therapeutic in the treatment of cardiovascular disease associated with arteriosclerosis and/or excessive LDL.

In various embodiments, the fusion construct comprises a polypeptide with about 50% or greater sequence identity to the ALK1 extracellular domain. In other embodiments, the polypeptide have equal to or greater than about 60%, 70%, 80%, 90%, 95%, 96%, 97%, 98%, 99% or 100% sequence identity with the ALK1 extracellular domain.

In certain embodiments, the fusion construct comprises a stability enhancing domain. The stability enhancing domain increases the stability of the construct in various ways, including but not limited to extending the construct's half-life, both in vivo or in vitro during storage or preparation, or increasing resistance to degradation. In other embodiments, the stability enhancing domain is the Fc region of an antibody. In yet other embodiments, the stability enhancing domain is IgG-Fc, thioredoxin or albumin.

In certain embodiments, albumin refers to human serum albumin. Usage of other albumins, such as bovine serum albumin, equine serum album and porcine serum albumin, are also contemplated within the invention.

In certain embodiments, the ALK1 extracellular domain and the stability enhancing domain are directly linked. In other embodiments, the ALK1 extracellular domain and the stability enhancing domain are linked through a polypeptide comprising 1-18 amino acids, 1-16 amino acids, 1-14 amino acids, 1-12 amino acids, 1-10 amino acids, 1-8 amino acids, 1-6 amino acids, 1-5 amino acids, 1-4 amino acids, 1-3 amino acids, 1-2 amino acids or a single amino acid.

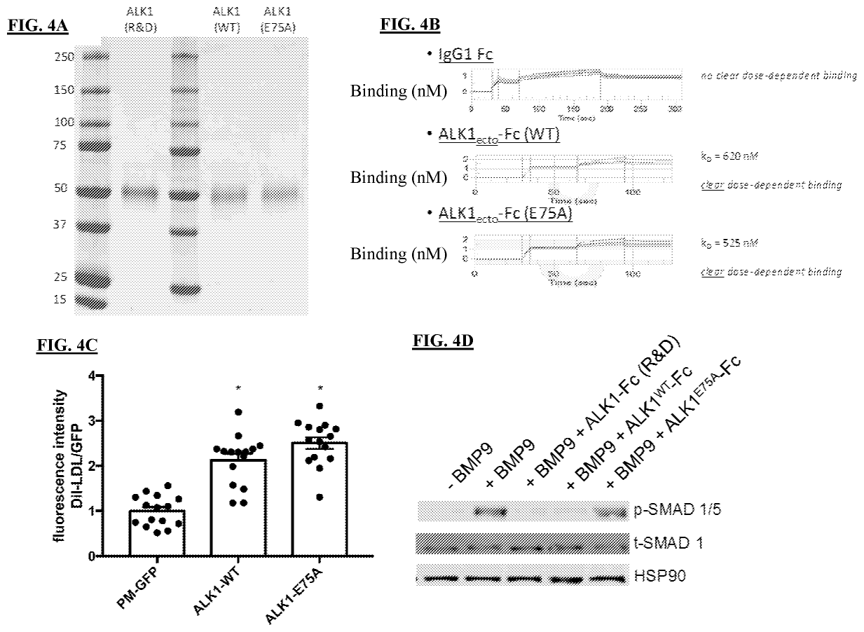

ALK1 possesses two binding sites that independently bind LDL and BMP9/10. In certain embodiments, disruption of BMP9/10 signaling is undesirable within the methods of the invention. In other embodiments, the fusion construct is altered to remove or minimize the ALK1 extracellular domain's capacity to specifically bind BMP9/10. In yet other embodiments, the ALK1 extracellular domain comprises an E75 point mutation (such as but not limited to E75A, E75F or E75V), and/or an H73 (such as but not limited to H73G, H73A, or H73D) point mutation. As shown in FIGS. 4A-4D and discussed in Example 3, this point mutation ablates ALK1's ability to specifically bind BMP9/10 without affecting its capacity to bind LDL.

In certain embodiments, the fusion construct is incorporated into a pharmaceutical composition that further includes at least one pharmaceutically acceptable carrier. Any pharmaceutical composition appropriate for the delivery of a biologic may be used, and various examples are described elsewhere herein or known in the art.

### Antibodies

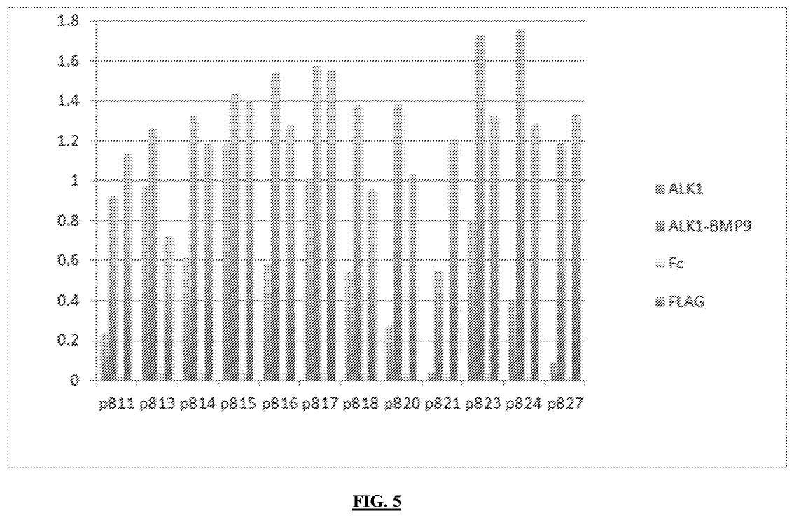

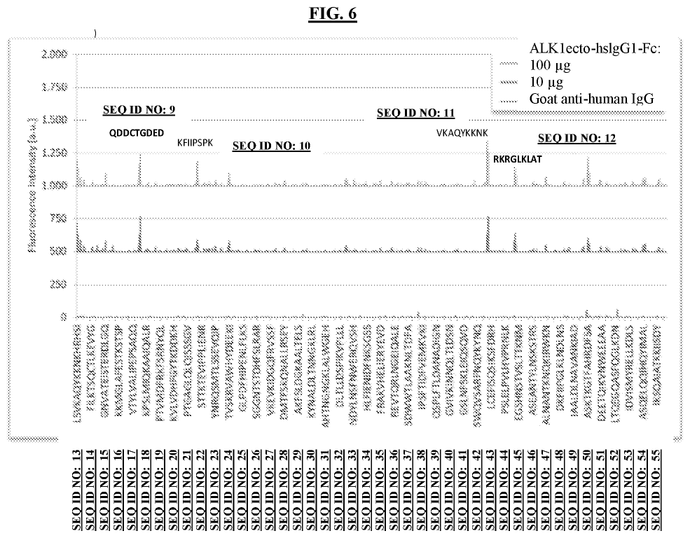

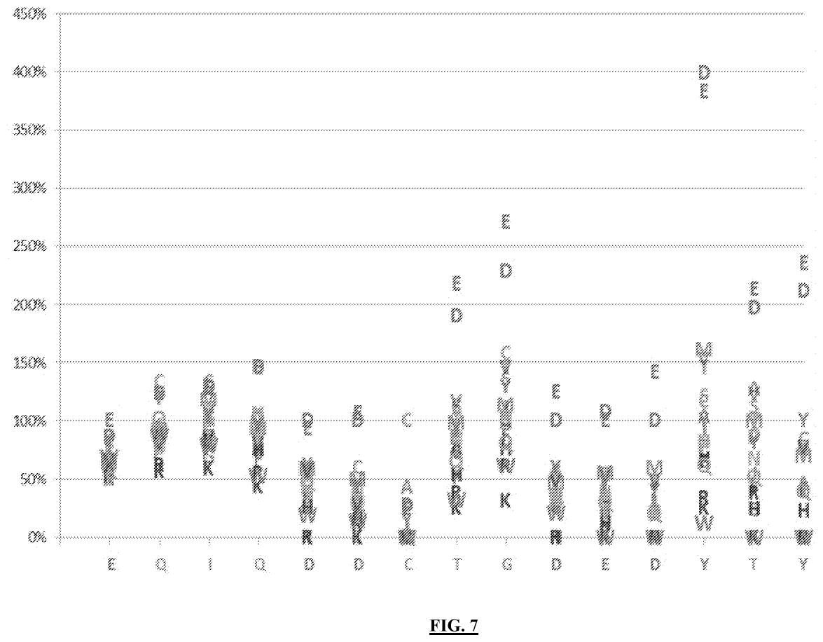

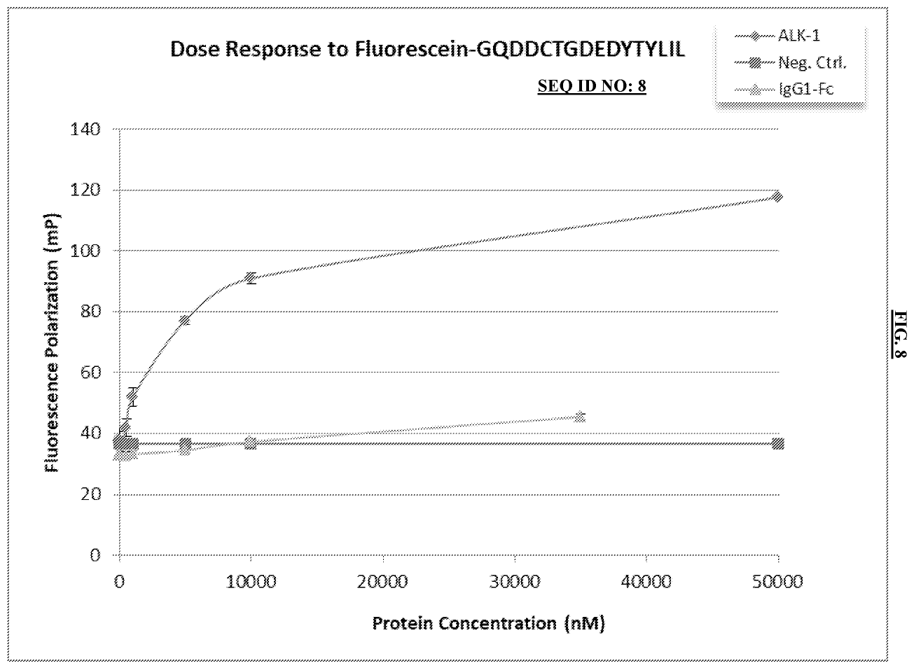

In another aspect, the invention comprises an antibody, such as a monoclonal antibody, that binds the ALK1 extracellular domain itself and ALK1 extracellular domain when complexed to BMP9/10. In certain embodiments, the antibody interferes with or blocks the ALK1-LDL interaction, and does not interfere with the ALK1-BMP9/10 interaction. As shown in FIG. 5 and discussed in Example 4, antibodies have been generated that block binding of LDL to ALK1 but do not interfere with the binding of BMP9/10 to ALK1. Without wishing to be limited by theory, the antibodies of the invention bind ALK1 in a manner that occludes the LDL binding site without disrupting ALK1 binding of BMP9/10.

In certain embodiments, the antibody recognizes and binds to a conformational epitope comprising amino acids 22-118 of ALK1.

The antibody of the invention binds to this epitope with a dissociation constant K_{d} equal to or less than about 10⁻⁶ M, about 10⁻⁷ M, about 10⁻⁸ M, about 10⁻⁹ M, about 10⁻¹⁰ M, or about 10⁻¹¹ M.

In various embodiments, the antibodies may be incorporated into a pharmaceutical composition that further includes at least one pharmaceutically acceptable carrier. Any pharmaceutical composition appropriate for the delivery of a biological agent can be used and various examples are described below.

As will be understood by one skilled in the art, any antibody that interferes with or blocks the ALK1 extracellular domain binding to LDL, but does not interfere with or block the ALK1 extracellular domain binding to BMP9/10, is useful in the present invention. The invention should not be construed to be limited to any one type of antibody, either known or heretofore unknown, provided that the antibody interferes with or blocks the ALK1 extracellular domain binding to LDL, but does not interfere with or block the ALK1 extracellular domain binding to BMP9/10. Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well-known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide can also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the invention should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), etc. Descriptions of techniques for preparing such monoclonal antibodies are well known and are described, for example, in Harlow et al., Antibodies: A Laboratory Manual, Cold Spring Harbor Laboratory, Cold Spring Harbor, N.Y. (1988); Harlow et al., Using Antibodies: A Laboratory Manual, (Cold Spring Harbor Press, New York, 1998); Breitling et al., Recombinant Antibodies (Wiley-Spektrum, 1999); and Kohler et al., 1997 Nature 256: 495-497; U.S. Pat. Nos. 5,693,762; 5,693,761; 5,585,089; and 6,180,370.

Nucleic acid encoding an antibody obtained using the procedures described herein can be cloned and sequenced using technology that is available in the art, and is described, for example, in Wright et al. (Critical Rev. in Immunol. 1992, 12:125-168) and the references cited therein. Further, the antibody of the invention may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody can be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. in Immunol. 1992, 12:125-168).

Processes such as those described above have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage that encode the Fab portion of an antibody molecule. However, the invention should not be construed to be limited solely to the generation of phage encoding Fab antibodies. Rather, phage that encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J Mol Biol 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

The invention encompasses polyclonal, monoclonal, synthetic antibodies, and the like. One skilled in the art would understand, based upon the disclosure provided herein, that the crucial feature of the antibody of the invention is that the antibody interferes with or blocks the ALK1 extracellular domain binding to LDL, but does not interfere with or block the ALK1 extracellular domain binding to BMP9/10.

### Methods

In another aspect, the invention comprises methods of reducing rates of LDL uptake in endothelial cells of a patient by administering the fusion construct or antibodies described above. In a further aspect, the invention comprises a method of treating arteriosclerosis or cardiovascular disease in a patient in need thereof by administering the fusion construct or antibodies described above.

In certain embodiments, the antibody or fusion construct can be administered in a pharmaceutical composition including at least one pharmaceutically acceptable carrier. In other embodiments, the method further comprises administering an additional treatment for cardiovascular disease. When the additional treatment for cardiovascular disease includes the administration of an additional agent, the additional agent can be administered separately or co-formulated in the pharmaceutical composition of the invention. In certain embodiments, the compositions of the invention are used in combination with any drugs approved for the treatment of atherosclerosis and familial hypercholesterolemia. In other embodiments, the additional treatment for cardiovascular disease is a lipid lowering agent. In yet other embodiments, the lipid lowering agent is an HMG-CoA reductase inhibitor, including but not limited to atorvastatin, cerivastatin, fluvastatin, lovastatin, mevastatin, pitavastatin, pravastatin, rosuvastatin, and simvastatin. In yet other embodiments, the additional treatment for cardiovascular disease is surgery, including but not limited to percutaneous coronary intervention, coronary artery bypass grafting and carotid endarectomy. In yet other embodiments, treatment may include plasmapheresis, inhibitors of Niemann-Pick C1-like 1 (e.g. ezetimibe), PCSK9 inhibitors (e.g. evolocumab), ApoB100 antisense molecules (e.g. mipomersen), MTP inhibtitor (e.g. lomitapide), ion exchange resins to inhibit the reabsorbtion of bile acid (e.g. colestyramine), fibrates (e.g. fenofibrate), niacin, or interference with intestinal absorbtion of cholesterol (e.g. β-sitosterol).

### Administration/Dosage/Formulations

Administration of the compounds and/or compositions of the present invention to a patient, preferably a mammal, more preferably a human, may be carried out using known procedures, at dosages and for periods of time effective to perform the method contemplated in the invention. An effective amount of the compound necessary for adequate binding of the compounds of the invention to their targets, respectively, may vary. One of ordinary skill in the art would be able to study the relevant factors and make the determination regarding the effective amount of the compound without undue experimentation.

Actual dosage levels of the active ingredients in the pharmaceutical compositions of this invention may be varied so as to obtain an amount of the active ingredient that is effective to achieve the required biological effect for a particular patient, composition, and mode of administration, without being toxic to the patient.

In certain embodiments, the compositions of the invention are formulated using one or more pharmaceutically acceptable excipients or carriers. In certain embodiments, the pharmaceutical compositions of the invention comprise an effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In certain embodiments, the dose of a compound of the invention is from about 1 ng to about 2,500 mg. In certain embodiments, a dose of a compound of the invention used in compositions described herein is less than about 10,000 μg, or less than about 8,000 μg, or less than about 6,000 μg, or less than about 5,000 μg, or less than about 3,000 μg, or less than about 2,000 μg, or less than about 1,000 oμg, r less than about 500 μg, or less than about 200 μg, or less than about 50 μg. Similarly, in certain embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other treatments for arteriosclerosis and/or cardiovascular disease.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans) buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 2003/0147952; 2003/0104062; 2003/0104053; 2003/0044466; 2003/0039688; and 2002/0051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

One in 10,000 people inherits homozygous mutations preventing the uptake of low-density lipoprotein (LDL) through the LDL receptor and causing hypercholesterolemia, which leads to atherosclerotic burden during adolescence and premature death. Atherosclerosis is initiated by subendothelial retention of lipoproteins. In humans, LDL is the main carrier of cholesterol to peripheral organs and is considered to be the major lipoprotein responsible for atherogenesis. LDL from the blood stream must cross the endothelium to reach the subendothelial area, where it can be retained and accumulate over time. At least 50% of LDL reaches the basolateral side of the endothelium in a pathway which can be blocked at 4° C., indicating an active transport mechanism. As atherosclerosis correlates tightly with the plasma LDL concentrations, plaque formation will occur in patients with elevated LDL. But, when LDL levels are elevated the main receptor for LDL the LDL receptor (LDLR) is down-regulated due to the excess of its ligand LDL. Therefore, the presence of an LDLR independent mechanism of LDL uptake in endothelial cells can be hypothesized. Electron microscopy of LDL transport across the endothelium in vivo indicates the existence of a concentration independent pathway for LDL transport from the endothelium to the intima. The pathway and receptor for the LDLR independent initial route of LDL permeation into the artery wall remain unknown. Paracellular leakage is unlikely responsible for the initiation of plaques, since LDL uptake, but not general permeability changes, occur in atheroprone areas of the vessel wall. This indicates the presence of an active transport mechanism for LDL, specifically expressed in lesion prone areas of the vasculature.

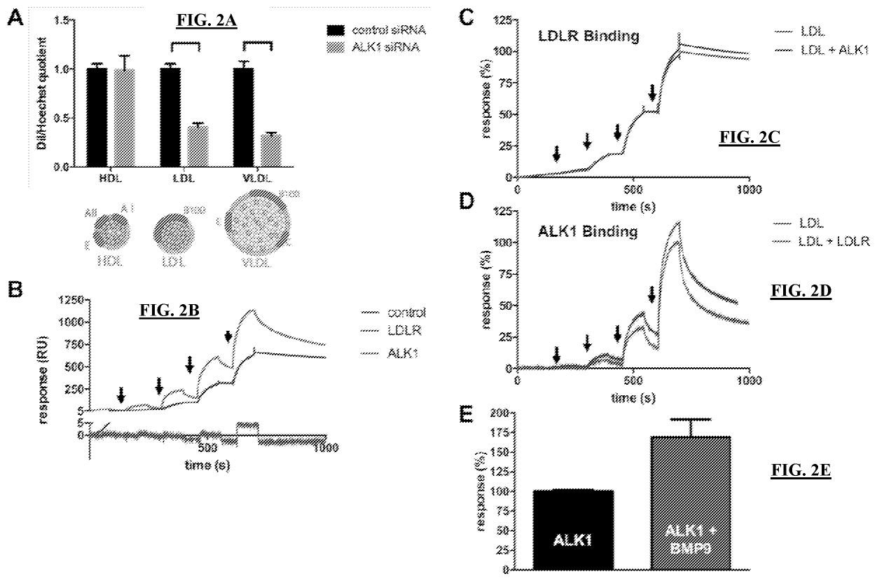

Studies were performed to elucidate unknown pathways that can promote LDL uptake into EC. Therefore, a high-throughput confocal microscopy-based assay examining fluorescently labeled LDL (DiI LDL) uptake was established using a genome wide RNAi library to target over 18,000 genes in endothelial cells (FIG. 2B). Primary gene hits were further analyzed for the effect on transferrin uptake to identify an LDL specific rather than a general endocytosis pathway. Remaining genes were analyzed in primary endothelial cells to rule out any artifacts of using a cell line. 34 genes were identified as immediately relevant for LDL uptake in endothelial cells. Only three of these genes (ALK1, ANGPT4, and GPR182) are expressed highly in the endothelium and are therefore preferential pharmacological targets as no other tissues would be affected (FIGS. 2A-2E).

The study revealed that Activin like kinase 1 (ALK1) mediates LDL uptake and transcytosis independently of the LDLR by direct binding to LDL in a non-degradative pathway. ALK1 is a type I serine/threonine receptor kinase as part of the transforming growth factor (TGF)-β superfamily, which has more than 30 members. The receptor is well-studied because mutations in the ACVRL1 gene can cause hereditary hemorrhagic telangiectasia (HHT), an autosomal dominant genetic disorder that leads to arteriovenous malformations (AVMs), abnormal blood vessel formation due to the lack of capillaries, but has not been linked to LDL metabolism before. The present study indicates that LDL uptake and transport via ALK1 is uncoupled from the biology of BMP9, HHT and AVM.

### Example 2

### Disruption of ALK1 Reduces Uptake of LDL

As seen in FIGS. 2A-2E, the loss of ALK1 via siRNA reduced the uptake of DiI-LDL and fluorescently labeled very low density lipoprotein (DiI-VLDL) uptake, but not fluorescently labeled high density lipoprotein (DiI-HDL) uptake, implying specificity for ApoB100 containing lipoproteins (FIG. 2A).

To directly examine protein-protein interactions between LDL and ALK1, surface plasmon resonance (SPR) was used with LDL immobilized to the chip and purified fragments of the Fc-tagged ectodomains for LDLR and ALK1 as analytes. As seen in FIG. 2B, specific binding of LDLR^{ecto} and ALK1^{ecto} was clear, with an apparent K_{d} of 7 nM and 200 nM for binding to LDLR and ALK1, respectively. To test if LDLR and ALK1 compete for binding to LDL, immobilized LDL was pre-bound with ALK1 or LDLR ectodomains, followed by the addition of the other ectodomain. The binding of LDLR^{ecto} was not altered when the LDL surface was pretreated with ALK1^{ecto} (FIG. 2C) or if LDL was pretreated with LDLR^{ecto} (FIG. 2D). These results indicate that LDLR and ALK1 bind to different sites on LDL.

ALK1^{ecto} binding to LDL was analyzed in the presence of equimolar concentrations of its cognate ligand, BMP9. ALK1^{ecto} binding was not perturbed by the presence of BMP9 (FIG. 2E) suggesting at least two distinct sites on the extracellular domain of ALK1 for binding each protein. Thus, these data demonstrate the success of the initial screen, and the utility of functional genomics to identify new cellular targets and pathways mediating LDL uptake in EC.

### Example 3

### ALK1 Decoy Receptor Does not Bind BMP9 but Neutralizes LDL

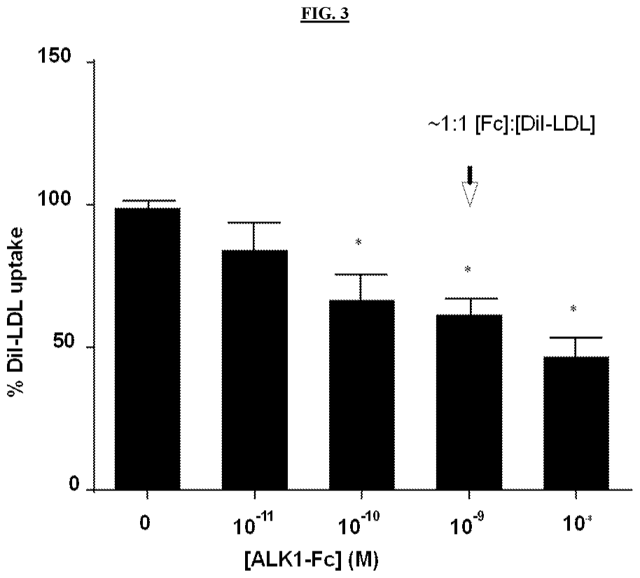

As seen in FIG. 3, ALK1-Fc blocks LDL uptake. Increasing concentrations of Fc proteins were incubated with endothelial cells for 60 min, followed by incubation with fluorescent DiI-LDL for an additional 60 minutes, and the amount of DiI-LDL taken up assessed by fluorescence assisted cell sorting (FACS) analysis. To avoid effects on angiogenesis, an E75A single point mutation was introduced into the extracellular domain of ALK1 in order to ablate its ability to bind BMP9/BMP10. A purification method yielded hundreds of micrograms of the decoy receptor in a highly-purified form (FIG. 4A). Bio-layer interferometry (BLI) showed that the E75A-mutant binds LDL to a similar extent than the wildtype form (FIG. 4B). Expressing the wildtype or the E75A mutant of full-length ALK1 in HeLa cells resulted in a significant increase in DiI-LDL uptake (FIG. 4C) and corroborates the finding that the E75A mutation does not influence the binding of ALK1 to LDL. The decoy receptor with E75A-mutation was tested to determine if it affects BMP9-signaling, and only the wildtype form inhibits BMP9-induced phosphorylation of phospho-SMAD1/5, while the E75A mutants lacks this ability and therefore will not affect angiogenesis (FIG. 4D).

### Example 4

### Antibodies

At least 12 antibodies (Fab fragments) have been selected against the BMP9/ALK1 complex (see FIG. 5 for ELISA data). Plates were coated with the 12 FLAG tagged antibodies, then incubated with Fc-ALK1, ALK1 precomplexed with BMP9 or Fc alone. Several antibodies bind ALK1 and the ALK1/BMP9 complex and based on SPR data, the binding site for LDL on ALK1 is distinct from where BMP9 binds ALK1. The antibodies detect the complex and/or bind to potential sites that disrupt LDL binding to ALK1.

The V_{H} and V_{L} regions of the 12 antibodies are cloned into an expression vector to produce full length Fc constructs in mammalian cells. Antibodies are tested with surface-plasmon resonance and competition assays with LDL and BMP9/BMP10. Similar to experiments above, antibodies are tested for blocking LDL uptake versus BMP9 signaling in endothelial cells. Any antibody that shows efficacy in such studies is affinity matured. In addition, the antibodies are crystalized with the ectodomain of ALK1. The antibody is also checked for its effect on retina vascular development in neonatal mice, which is a phenotype very sensitive to the levels of BMP9. In certain embodiments, the antibodies of the invention are useful for treating or preventing atherosclerosis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A fusion construct comprising a polypeptide with greater than 70% sequence identity to the ALK1 extracellular domain (SEQ ID NO: 1), which is fused to a stability enhancing domain, wherein the fusion construct does not bind BMP9 and/or BMP10.

2. The fusion construct of claim 1, wherein the polypeptide has greater than 90% sequence identity to the ALK1 extracellular domain.

3. The fusion construct of claim 1, wherein the ALK 1 extracellular domain comprises a point mutation in E75 and/or H73.

4. The fusion construct of claim 3, wherein the ALK1 extracellular domain comprises at least one point mutation selected from the group consisting of E75A, E75F and E75V.

5. The fusion construct of claim 3, wherein the ALK1 extracellular domain comprises at least one point mutation selected from the group consisting of H73G, H73A and H73D.

6. The fusion construct of claim 1, wherein the stability enhancing domain comprises albumin, thioredoxin or a Fc region of an antibody.

7. The fusion construct of claim 6, wherein the antibody is IgG1.

8. A pharmaceutical composition comprising the fusion construct of claim 1, and at least one pharmaceutically acceptable carrier.

9. A method of reducing, preventing or reversing LDL uptake into endothelial cells of a subject, the method comprising administering to the subject the fusion construct of claim 1.

10. The method of claim 9, wherein the subject is further administered an additional treatment for cardiovascular disease.

11. The method of claim 10, wherein the additional treatment for cardiovascular disease comprises administration of at least one HMG-CoA inhibitor.

12. A method of treating a LDL-related disease or disorder in a subject, the method comprising administering to the subject the fusion construct of claim 1.

13. The method of claim 12, wherein the subject is further administered an additional treatment for cardiovascular disease.

14. The method of claim 13, wherein the additional treatment for cardiovascular disease comprises administration of at least one HMG-CoA inhibitor.

15. The method of claim 12, wherein the disease or disorder includes at least one selected from the group consisting of familial hypercholesterolemia (FH) and atherosclerosis.

16. The method of claim 9, wherein the subject is a mammal.

17. The method of claim 16, wherein the mammal is human.
